(19) United States
(12) Patent Application Publication
(10) Pub. No.: US 2026/0021324 A1
(43) Pub. Date: Jan. 22, 2026

(54) Sleeve-Type Radioactive Source Delivery Assembly And Its Method of Use

(71) Applicant: HANGZHOU DASHTECH CO., LTD, Hangzhou (CN)

(72) Inventors: Xuetang Wang, Hangzhou (CN); Dingchen Zhu, Hangzhou (CN); Guangming Fu, Hangzhou (CN); Xingxing Lei, Hangzhou (CN)

(21) Appl. No.: 19/259,854

(22) Filed: Jul. 3, 2025

Related U.S. Application Data
(63) Continuation of application No. PCT/CN2023/140955, filed on Dec. 22, 2023.

(30) Foreign Application Priority Data
Jan. 5, 2023 (CN) 2023100147077
Jun. 1, 2023 (CN) 2023106384729
Aug. 11, 2023 (CN) 2023110124955
Oct. 18, 2023 (CN) 2023113489176

(51) Int. Cl. A61N 5/10

(52) U.S. Cl. CPC A61N 5/1007; A61N 2005/101; A61N 2005/1011; A61N 2005/1024

(57) ABSTRACT

This present invention discloses a sleeve-type radioactive source delivery assembly and its method of use. The outer tube is sleeved over the outside of the inner tube, the front end of the inner tube is connected to a puncture needle that is inserted into the target body, and the front end of the outer tube abuts against or connects to the target body. The front end of the puncture needle is provided with a restrictive segment. when an external force is applied to the radioactive source, the restrictive segment is expanded by the radioactive source, allowing the radioactive source to pass through. The inner tube and the outer tube are capable of relative movement under the action of a needle pulling driver, so that the puncture needle moves in a direction away from the target body and is withdrawn from the target body.

This application is a continuation of International Patent Application No. PCT/CN2023/140955, filed on Dec. 22, 2023, which claims priority of Chinese Patent Application No. 202310014707.7, filed on Jan. 5, 2023, No. 202311012495.5, filed on Aug. 11, 2023, No. 202310638472.9, filed on Jun. 1, 2023, No. 202311348917.6, filed on Oct. 18, 2023, the entire contents of each of which are hereby incorporated by reference.

### FIELD

This invention relates to the field of medical devices, and more specifically, to a sleeve-type radioactive source delivery assembly and its method of use.

### BACKGROUND

Brachytherapy involves percutaneously inserting multiple radioactive seeds directly into the tumor to perform localized radiotherapy. This procedure is applicable to a wide range of tumors, including lung cancer, liver cancer, breast cancer, prostate cancer, etc. It offers the advantages of small incisions, minimal bleeding, and relatively few surgical complications while effectively inhibiting tumor growth.

The procedure is as follows: firstly, puncture needles are inserted into the tumor assisted by a puncture guidance template, ensuring that the spacing and orientation among needles are consistent with the preoperative plan. After confirming via imaging that all puncture needles are in place, the doctor uses the channel created by puncture needle to implant seeds-advancing one seed, pulling the needle trocar partly out of the target body, advancing another seed, pulling the needle trocar partly out of the target body, and repeating this process until all planned seeds are implanted.

However, this pattern is time-consuming, and doctors are faced with radiation exposure for handling radioactive seeds, which greatly limits the application and promotion of such treatment. Therefore, there is an urgent need for a flexible delivery system for radioactive source that enables automatic needle pulling and seed delivery, avoiding radiation exposure and allowing multiple seeds to be sequentially delivered through the needle in one-time to improve implantation efficiency.

### SUMMARY

The invention is intended to provide a sleeve-type radioactive source delivery assembly and its method of use. The inner tube and outer tube could move relatively by the needle pulling driver of the radioactive source implantation machine, which enables automatic puncture needle withdrawal and the seed delivery, thereby avoiding radiation exposure.

To achieve the above objective, the invention provides the following technical solution:
a sleeve-type radioactive source delivery assembly, comprising an inner tube and an outer tube, wherein the outer tube is sleeved over the inner tube, the front end of the inner tube is connected to a puncture needle for insertion into the target body, and the front end of the outer tube abuts against or is connected to the target body. The inner tube and the outer tube can move relatively by the needle pulling driver, so that the puncture needle withdraws from the target body.

Preferably, the outer tube is made of a flexible and deformable material such as plastic. The inner tube has an inner diameter of 0.5-1.5 mm and an outer diameter of 1.5-5 mm, and the inner diameter of the outer tube is larger than the outer diameter of the inner tube.

Preferably, the portion of the inner tube near the puncture needle is made of a flexible and deformable material such as plastic, and the portion away from the puncture needle is made of a rigid material with a yield strength greater than 50 MPa.

Preferably, the inner tube is directly connected to the puncture needle, which is sleeved over the front segment of the inner tube and bonded in place. The front of the inner tube is sleeved with a rigid guiding tube, and the rear portion of the inner tube is a pushing guiding tube.

Preferably, the end of the inner tube is provided with a quick-connection structure A, which may be one or more of threaded connection, snap-fit connection, or adhesive bonding. When the quick-connection structure A is snap-fit connection, the front end of the inner tube forms snap-fit A as the quick-connection structure A, and the rear end of the puncture needle forms a matching snap-fit B. A locking sleeve A is sleeved over the inner tube and, when slid to the position where snap-fit A and snap-fit B engage, locks the inner tube and the puncture needle together.

Preferably, when snap-fit A is fixed on the front end of the inner tube, markings are provided on the front and rear outer surfaces of the inner tube and on the rear outer surface of the puncture needle.

Preferably, the front end of the inner tube connects to the puncture needle via a free-spinning connection structure consisting of a fixed sleeve and a free-spinning connector. The fixed sleeve is sleeved on the front end of the inner tube and has a step that forms a rotation slot with the front face of the inner tube. The rear end of the free-spinning connector has a shaft portion that fits into the rotation slot, allowing rotation. The front end of the free-spinning connector is provided with a quick-connection structure B, which may be one or more of threaded connection, snap-fit connection, or adhesive bonding. For snap-fit connection, the free-spinning connector has snap-fit C, and the puncture needle has a matching snap-fit D, with locking sleeve B used for locking.

Preferably, the outer tube comprises an outer tube segment and a middle adjustment segment, with the middle adjustment segment sleeved outside the outer tube segment.

Preferably, the outer tube segment includes a main tube segment and an adjustment tube segment, the adjustment tube segment is sleeved outside the main tube segment, and relative movement and fixation between the adjustment tube segment and the main tube segment allows adaptive adjustment of the total effective length of the outer tube to match different insertion depths of puncture needle. The adjustment tube segment and the main tube segment may be integrally formed or connected, and the adjustment tube segment is a second adjustment tube.

Preferably, a pushing tube base is provided at the rear end of the second adjustment tube away from the target body. The pushing tube base has a pushing surface with an outer diameter or circumscribed circle diameter greater than 10 mm to increase the contact area with the needle pulling pusher.

Preferably, a locking structure is provided at the front end of the second adjustment tube near the puncture needle to fix its position relative to the main tube segment.

Preferably, when the second adjustment tube and the outer tube segment move towards each other to reduce the effective length of the outer tube, the connection portion between the inner tube and puncture needle is exposed from within the outer tube.

Preferably, a plurality of stop steps is provided along the longitudinal outer surface of the main tube segment. The stop steps may be one or a combination of annular slots, snap holes, or protrusions. The locking structure includes a limit head, such as an elastic snap block, a snap-fit, a latch, or a screw, that engages with a limit step to fix the second adjustment tube and main tube segment relative to each other.

Preferably, when the stop steps are annular slots, the adjustment tube segment is an annular slot tube with a linear array of annular slots spaced 1-10 mm apart. The locking structure is an elastic snap-fit latch, and its latch is locked into an annular slot to fix the adjustment tube segment.

Preferably, the front end of the puncture needle is provided with a restrictive segment with a maximum inscribed circle diameter smaller than the outer diameter of the radioactive source, allowing the radioactive source to be blocked by gravity alone, and only under an external force does the restrictive segment expand and allow the source to pass.

Preferably, the restrictive segment is formed by one or a combination of a concave capillary tube, a variable diameter segment, or resistance fillers. The resistance filler may be a thin sheet, wire, or elastic body such as polyurethane, silicone, latex, or rubber.

A method for using a sleeve-type radioactive source delivery assembly includes: inserting the puncture needle into the target body; connecting and fixing the front end of the inner tube to the rear end of the puncture needle, or inserting the puncture needle pre-assembled with the inner tube; abutting the front end of the outer tube against the target body; fixing the rear end of the inner tube to a radioactive source implantation machine; using the needle pulling driver to move the outer tube relative to the inner tube, So that the puncture needle withdraw from the target body.

Preferably, the main tube segment of the outer tube is pushed towards the target body to make its front end abut the target body, and then the second adjustment tube is pulled back relative to the main tube segment to increase the total effective length of the outer tube, after which the locking structure fixes the relative position between the main tube segment and the second adjustment tube.

Preferably, after fixing the rear end of the inner tube to the radioactive source implantation system, the needle pulling pusher pushes the pushing tube base to drive the outer tube toward the target body, achieving puncture needle withdrawal.

Preferably, multiple radioactive sources are pushed simultaneously through the inner tube using a flexible pushing wire. As the first seed is delivered to the target body, the needle pulling driver drives the inner tube to move relative to the outer tube, withdrawing the puncture needle; after withdrawing by a preset distance, the second seed is pushed by the flexible pushing wire, repeating the process until all seeds are implanted according to the plan.

Beneficial effects of the invention are as follows:

1. The outer tube is sleeved over the inner tube, and under the action of the needle pulling driver, relative movement between the tubes causes the puncture needle to be pulled from the target body. It can change the implantant position of the radioactive source within the target body. The adjustment tube segment enables adaptation to different needle insertion depths, ensuring sufficient withdrawal stroke.

2. The restrictive segment at the puncture needle tip allows accumulation of multiple radioactive seeds without falling out, improving the implant efficiency.

3. The quick-connection structure or free-spinning connection structure allows fast and reliable connection/disconnection between the puncture needle and the inner tube.

### DRAWING DESCRIPTION

FIG. 1 is a schematic diagram of the overall structure of the delivery assembly in Example 1;

FIG. 2 is a segmental structure schematic diagram of FIG. 1;

FIG. 3 is an enlarged structure schematic diagram of part A in FIG. 2;

FIG. 4 is an enlarged structure schematic diagram of part B in FIG. 2;

FIG. 5 is an enlarged structure schematic diagram of part C in FIG. 2;

FIG. 6 is an enlarged structure schematic diagram of part D in FIG. 2;

FIG. 7 is an enlarged structure schematic diagram of part E in FIG. 2;

FIG. 8 is an enlarged structure schematic diagram of part F in FIG. 2;

FIG. 9 is a schematic diagram of the structure of the needle stylet with a conical head at the front end and the needle trocar;

FIG. 10 is a schematic diagram of the structure of the needle stylet in FIG. 9;

FIG. 11 is a schematic diagram of the structure of the needle trocar in FIG. 9;

FIG. 12 is a segmental view of the needle trocar in FIG. 9;

FIG. 13 is a schematic diagram of the structure of Example 2;

FIG. 14 is a front view of Example 2;

FIG. 15 is a segmental view of A-A in FIG. 14;

FIG. 16 is a schematic diagram of the structure of the outer tube;

FIG. 17 is a segmental view of B-B in FIG. 16;

FIG. 18 is a schematic diagram of the pushing tube base in Example 2;

FIG. 19 is a segmental view of D-D in FIG. 18;

FIG. 20 is a schematic diagram of the structure of Example 3;

FIG. 21 is a segmental view of FIG. 20;

FIG. 22 is an enlarged schematic diagram of the locking mechanism in Example 3;

FIG. 23 is a schematic diagram of the structure of Example 4;

FIG. 24 is an enlarged schematic diagram of the locking mechanism in Example 4;

FIG. 25 is a schematic diagram of the structure of Example 5;

FIG. 26 is a schematic diagram of the structure when the inner tube and the puncture needle are connected through the locking mechanism in Example 7;

FIG. 27 is a first disassembled structure schematic diagram when the inner tube and the puncture needle are connected through the locking mechanism in Example 7;

FIG. 28 is a second disassembled structure schematic diagram when the inner tube and the puncture needle are connected through the locking mechanism in Example 7;

FIG. 29 is an internal schematic diagram when the inner tube and the puncture needle are connected through the locking mechanism in Example 7;

FIG. 30 is a schematic diagram of the structure when the inner tube and the puncture needle are connected through the free-spinning structure in Example 8;

FIG. 31 is a schematic diagram of the structure without the locking sleeve B in Example 8;

FIG. 32 is a segmental view of the connection structure of the inner tube and the puncture needle through the free-spinning structure in Example 8;

FIG. 33 is a schematic diagram of the structure of the free-spinning structure in Example 8.

### DETAILED EMBODIMENTS

In the following, the technical solutions of the embodiments of the present invention will be described in detail with reference to the accompanying drawings, clearly, the described embodiments are only part of the present invention and not all of them. Based on the embodiments herein, all other embodiments obtained by those of ordinary skill in this field without creative efforts fall within the scope of protection of the present invention.

### Embodiment 1

A sleeve-type radioactive source delivery assembly includes the inner tube (e.g., the first inner tube 204301 of this embodiment) and the outer tube (e.g., the first outer tube 204302), the outer tube is sleeved over the inner tube, the front end of the inner tube is connected to a puncture needle inserted into a target body (e.g., the needle trocar 204303 of this embodiment), and the front end of the outer tube abuts or connects to the target body; under the action of the needle pulling driver, the inner and outer tubes can move relative to each other, so that the puncture needle moves in a direction away from the target body and is withdrawn from it. The target body may include biological tissue, a base, or a puncture guidance template.

The outer tube is made of a flexible, bendable, and deformable plastic material, the portion of the inner tube near the puncture needle is also made of such plastic material. The inner diameter of the inner tube is 0.5-1.5 mm and the outer diameter is 1.5-5 mm. The inner diameter of the outer tube is greater than the outer diameter of the inner tube.

The inner tube may be directly connected to the puncture needle; or the front end of the inner tube may be provided with a connector a for quick connection to the puncture needle, where the connection method includes one or more of, threaded connection, snap-fit connection, or adhesive bonding.

When the inner tube is directly connected to the puncture needle, the puncture needle is inserted into the front portion of the inner tube and bonded to it. The rigid guiding tube (e.g., rigid guiding tube A204306) is sleeved over the front portion of the inner tube, and the rear portion of the inner tube is the pushing guiding tube (e.g., rigid guiding tube B204307). The outer tube includes the outer tube segment and the middle adjustment segment. The middle adjustment segment is slidably sleeved onto the outer tube segment.

The outer tube segment comprises a main tube segment (e.g., the first main tube segment 2043021) and an adjustment tube segment (e.g., the first adjustment tube segment 2043023), the adjustment tube segment is located on rear side of the main tube segment, the middle adjustment segment is sleeved over the outside of the adjustment tube segment, and is controlled to move relative to and lock against the adjustment tube segment so that the total effective length of the outer tube can be adaptively adjusted according to different insertion depths of the needle.

The main tube segment and the adjustment tube segment are integrally formed or connected.

When the second adjustment tube and the adjustment tube segment move toward each other to minimize the total effective length of the outer tube, the connecting portion between the inner tube and the puncture needle becomes exposed from within the outer tube.

The second adjustment tube is provided with the locking mechanism (e.g., locking mechanism 2043082) at the front end. The locking mechanism is used to fix the relative position between the second adjustment tube and the adjustment tube segment.

The adjustment tube segment has multiple stop steps on the outer surface along the length. These stop steps may be annular slots (e.g., annular slot 20430231), snap holes, protrusions, or a combination thereof; the locking mechanism includes a stop head that engages with the stop steps and locks the relative position of the second adjustment tube and adjustment tube segment, the stop head may be one or more of: an elastic snap block, a snap-fit, a latch, or a screw.

When the stop steps are annular slots, the adjustment tube segment is an annular slot tube with a linear array of multiple annular slots spaced 1-10 mm apart, the locking mechanism is an elastic snap-fit equipped with a latch that can lock into the annular slot to secure the second adjustment tube and adjustment tube segment.

The pushing tube base (e.g., the first pushing tube base 2043083) is provided at the rear end of the second adjustment tube away from the target body, the pushing tube base has a pushing surface (e.g., pushing surface 20430831) at the rear end, and its outer diameter or circumscribed diameter is greater than 10 mm, thereby increasing contact area with the needle pulling pusher.

The front end of the puncture needle is provided with a restrictive segment, the maximum inscribed circle diameter of which is smaller than the outer diameter of the radioactive source, the radioactive source cannot pass through the restrictive segment of the puncture needle by gravity alone, thereby preventing the radioactive source from falling; when an external force is applied to the radioactive source, the restrictive segment is expanded by the radioactive source, thereby allowing the radioactive source to pass.

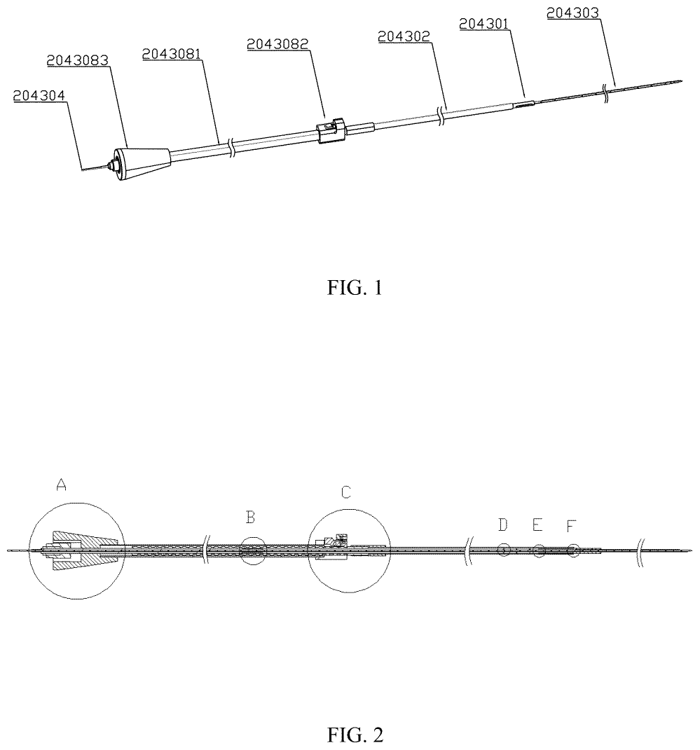

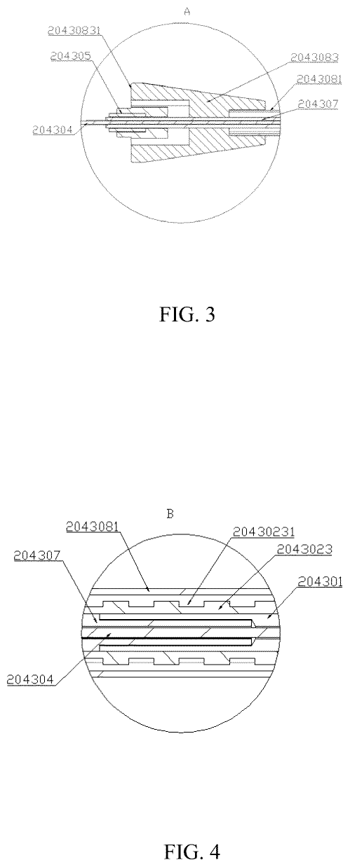

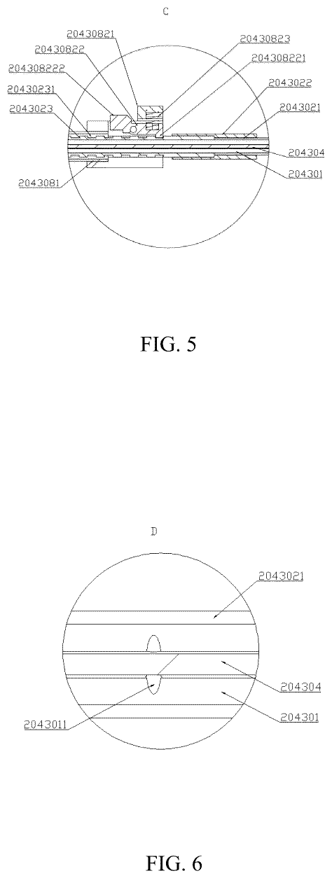

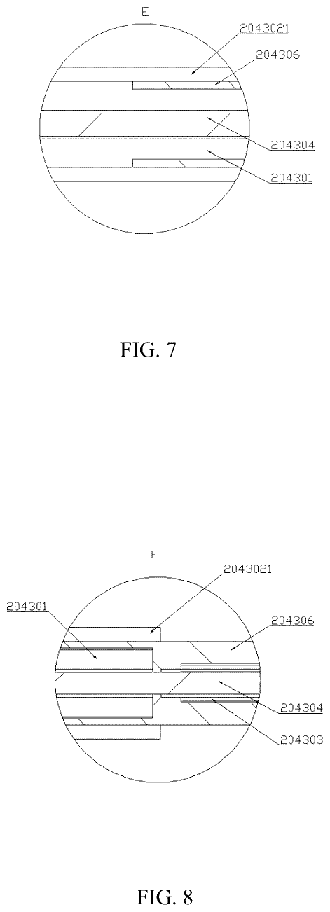

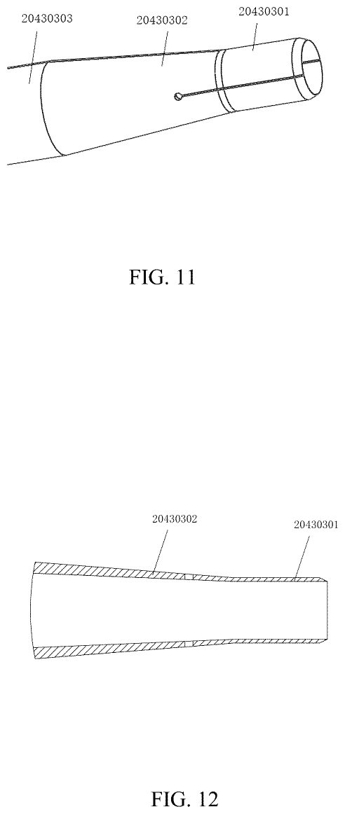

As shown in FIGS. 1 to 12, the outer tube is adjustably sleeved over the inner tube. The specific structure is as follows:

The front end of the first inner tube 204301 is connected to the needle trocar 204303, and the rear end of the first inner tube 204301 is connected to the first inner tube connector 204305, which connects to the external docking plate, the first outer tube 204302 is sleeved over the outside of the first inner tube 204301, with adjustable length, a first needle stylet 204304 is disposed inside the first inner tube 204301, and the front end of the first needle stylet 204304 is either flush with or slightly protrudes from the front end of the needle trocar 204303.

The first inner tube 204301 is made of a flexible, bendable, and deformable material, specifically plastic. Its inner diameter is 0.5-1.5 mm, and its outer diameter is 1.5-5 mm.

The first outer tube 204302 includes a first main tube segment 2043021, a connecting tube segment 2043022, and a first adjustment tube segment 2043023, the connecting tube segment 2043022 is sleeved over both the first main tube segment 2043021 and the first adjustment tube segment 2043023, and is bonded and fixed.

The first adjustment tube segment 2043023 is sleeved with a pushing tube 2043081, which can be moved relative to and locked against the first adjustment tube segment 2043023, this allows the total effective length of the first outer tube 204302 and the pushing tube 2043081 (regarded as the outer tube assembly) to be adaptively adjusted based on different insertion depths of the needle. The pushing tube 2043081 is equipped with a locking mechanism 2043082 for fixing its relative position with respect to the first adjustment tube segment 2043023, the first adjustment tube segment 2043023 has multiple annular slots 20430231 along its outer surface in the length direction. The locking mechanism 2043082 includes a pushing tube front seat A 20430821, a snap-fit member 20430822, and a spring 20430823, The front seat A 20430821 is sleeved over the front end of the pushing tube 2043081 and bonded in place, the snap-fit member 20430822 is rotatably mounted on the front seat A 20430821. One end of the snap-fit member 20430822 is a snap-fit portion 204308221, and the other end is an operating portion 204308222, the spring 20430823 is positioned between the snap-fit member 20430822 and the front seat A 20430821, providing a forward rotational elastic force to the snap-fit member, this causes the snap-fit portion 204308221 to engage into an annular slot 20430231, the operating portion 204308222 extends out from the front seat A. When adjusting the position of the pushing tube 2043081, pressing the operating portion 204308222 rotates the snap-fit member in reverse to release the snap-fit portion from the current annular slot, then, the pushing tube 2043081 is slid to the desired position. Once the pressing is released, the spring 20430823 drives the snap-fit portion 204308221 to re-engage into the corresponding annular slot 20430231, thereby locking the position of the pushing tube 2043081 relative to the first adjustment tube segment 2043023, the spring 20430823 may also be replaced with other types of elastic structures.

The rear end of the pushing tube 2043081 is sleeved with the first pushing tube base 2043083, the pushing tube base 2043083 has a pushing surface 20430831 at its end away from the puncture needle, the outer diameter or circumscribed diameter of the pushing surface 20430831 is greater than 10 mm, thereby increasing the contact area with the needle pulling pusher.

The front end of the needle trocar 204303 is equipped with a reduced-diameter segment, the inner diameter of which is smaller than the outer diameter of the radioactive source, the outer wall of the needle trocar 204303 is marked with a scale, enabling the user to observe the insertion depth.

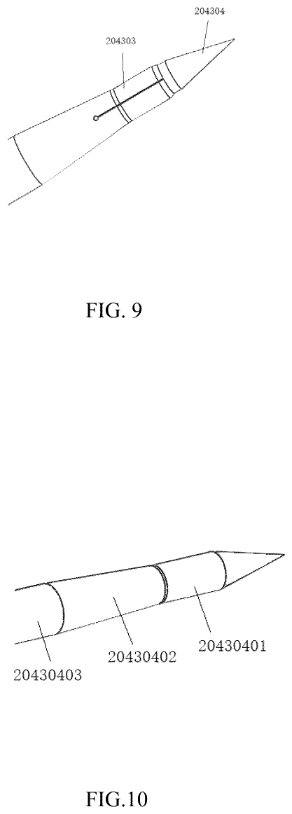

As shown in FIGS. 9 to 12, preferably, when the front end of the first needle stylet 204304 is a conical head, its rear end is provided, in the direction away from the cone, with a small-diameter segment 20430401, a tapered transition segment 20430402, and a large-diameter segment 20430403, the needle trocar 204303 is provided with a small-diameter segment 20430301, a tapered transition segment 20430302, and a large-diameter segment 20430303, the inner diameter of the small-diameter segment is smaller than the outer diameter of the radioactive source, the small-diameter segment and the tapered transition segment of the needle trocar are provided with slits to ensure that the front end of the needle trocar can elastically open and close, the slit extends from the end face of the small-diameter segment of the needle trocar to the tapered transition segment. At the end of the slit is an anti-stress concentration slit hole, the diameter of which is greater than the width of the slit. The wall thickness of the inner surface of the tapered transition segment 20430302 gradually thins from the large-diameter segment 20430303 toward the small-diameter segment 20430301, or the entire region from the large-diameter segment 20430303 to the distal end is thinned. The purpose of this is to reduce the rigidity of the needle trocar 204303 and improve its elasticity, so that the force to push the seed out of the needle trocar 204303 would not be too large.

### Embodiment 2

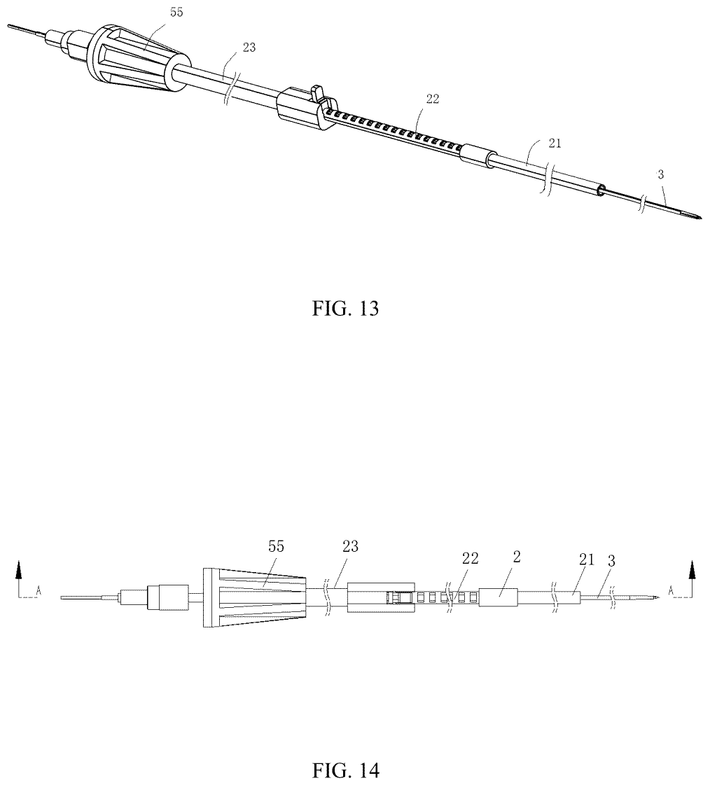

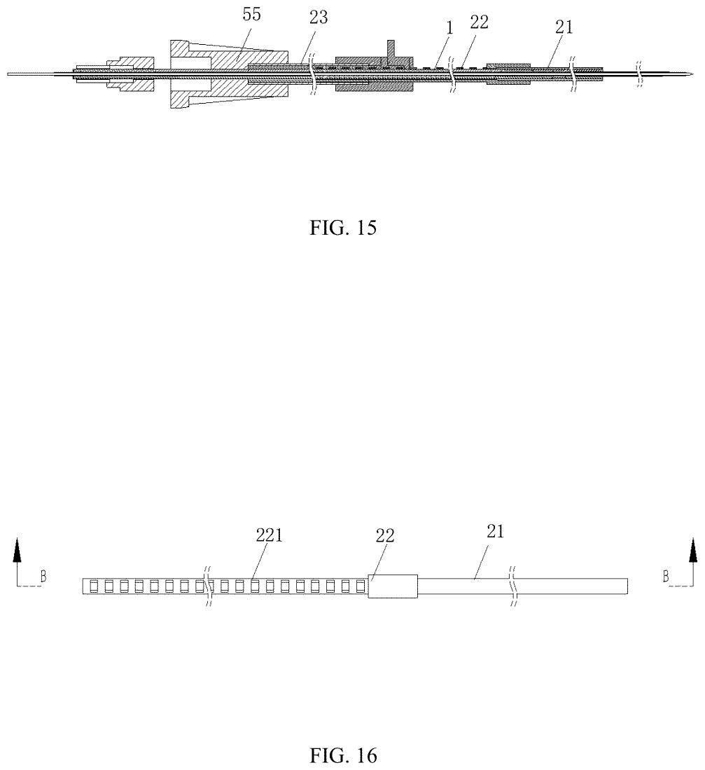

As shown in FIGS. 13 to 19, a sleeve-type radioactive source delivery assembly includes a second inner tube 1 and a second outer tube 2, the second outer tube 2 is sleeved over the outside of the second inner tube 1, the front end of the second inner tube 1 is connected to a puncture needle 3 that is inserted into the target body, and the front end of the second outer tube 2 abuts against or connects to the target body; the second inner tube 1 and the second outer tube 2 are capable of relative movement under the action of the needle pulling driver, so that the puncture needle moves away from the target body and is withdrawn from it.

The second outer tube 2 is made of a flexible, bendable, and deformable material, specifically plastic, the second inner tube 1 is also made of such a plastic material, the inner diameter of the second inner tube is 0.5-1.5 mm, and the outer diameter is 1.5-5 mm, the inner diameter of the second outer tube is greater than that of the second inner tube, and the second outer tube is made of a transparent material, the second inner tube 1 and/or the puncture needle 3 is provided with markings, which can be observed through the second outer tube 2 to view the second inner tube 1 and the puncture needle 3.

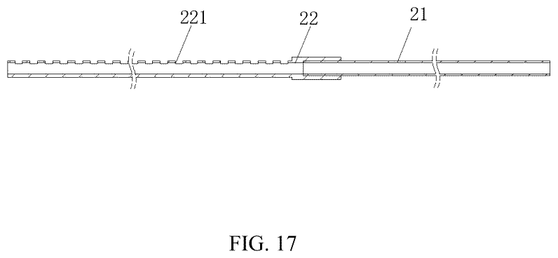

As shown in FIGS. 16 to 17, the second outer tube includes an outer tube segment and a middle adjustment segment, the middle adjustment segment is slidably sleeved and mounted on the outer tube segment.

The second outer tube 2 includes a main tube segment 21 and a adjustment tube segment 22, the adjustment tube segment 22 is arranged on one side of the main tube segment, the middle adjustment segment is sleeved over the outside of the adjustment tube segment 22, it is controlled to move relative to the adjustment tube segment and locked, so that the total effective length of the second outer tube can be adaptively adjusted based on different insertion depths of the needle body, this allows the needle pulling driver to drive the second inner tube 1 and second outer tube 2 to perform sufficient relative movement, withdrawing the puncture needle from the target body by a sufficient length.

The main tube segment 21 and the adjustment tube segment 22 are integrally formed or connected. In this embodiment, the main tube segment 21 and the adjustment tube segment 22 are bonded together.

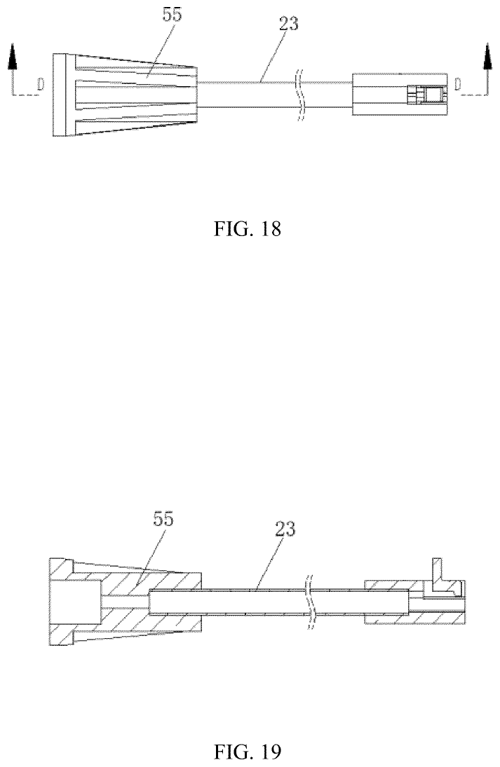

As shown in FIGS. 18 to 19, the middle adjustment segment is a second adjustment tube 23, the second adjustment tube is sleeved on the outside of the adjustment tube segment, it is controlled to move relative to the adjustment tube segment so that the total effective length of the second outer tube and the second adjustment tube can be adaptively adjusted according to the insertion depth of the needle; the second adjustment tube 23 is provided with a locking mechanism at the front end, which is used to fix the relative position between the second adjustment tube and the adjustment tube segment.

The adjustment tube segment has multiple stop steps along its outer surface in the length direction, these stop steps may be one or a combination of slots, snap holes, or protrusions. In this embodiment, the stop steps use slots 221.

The locking mechanism includes a stop head that mates with the stop steps to fix the relative position between the second adjustment tube 23 and the adjustment tube segment 22, the stop head may be one or more of: an elastic snap block, a snap-fit, a latch, or a screw.

The second adjustment tube 23 is provided with a second pushing tube base 55, the second pushing tube base is provided at the end of the second adjustment tube that is farthest from the target body, the rear end of the second pushing tube base is provided with a pushing surface, the outer diameter or circumscribed diameter of the pushing surface is greater than 10 mm, thereby increasing the contact area with the needle pulling pusher.

### Embodiment 3

The parts of this embodiment that are the same as in Embodiment 2 will not be repeated here. The differences are as follows:

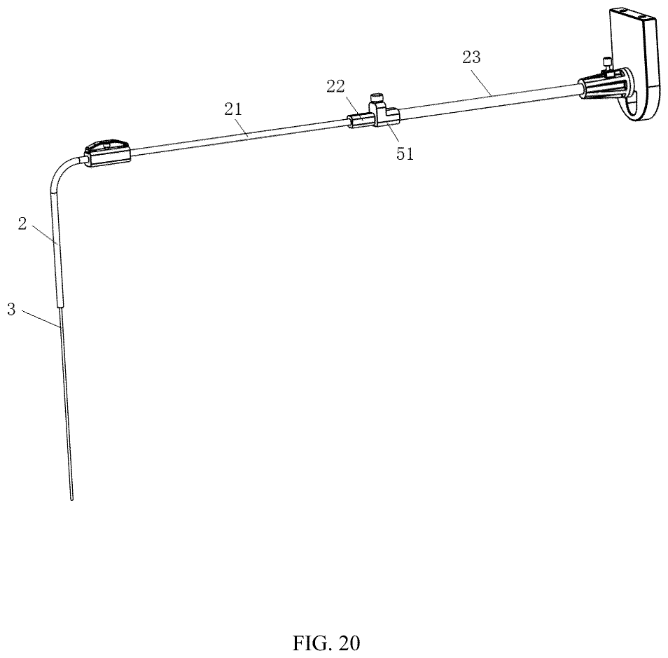

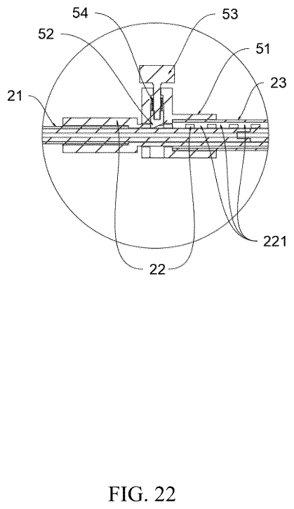

As shown in FIGS. 20 to 22, the second outer tube 2 is sleeved on the outside of the second inner tube 1, and the front end of the second inner tube 1 is connected to the puncture needle 3 inserted into the target body. The front end of the second outer tube 2 is either in contact with or connected to the target body. The main tube segment 21 and the adjustment tube segment 22 are bonded together, and the locking mechanism includes a stop head that cooperates with the stop step to fix the relative position between the second adjustment tube 23 and the adjustment tube segment 22.

When the stop head is a buckle, the locking mechanism includes: a second push tube front seat 51, a buckle 52, a first adjustment screw 53, and a first spring 54.

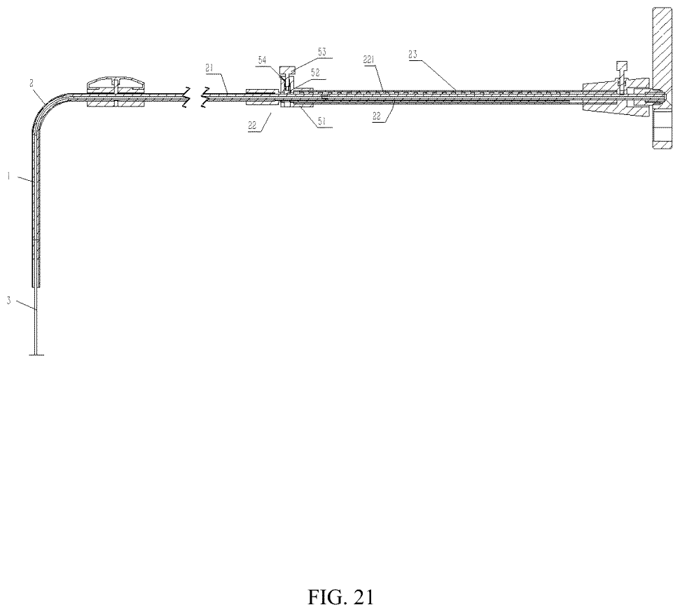

As shown in FIG. 21, the second pusher tube front seat 51 is slidably sleeved on the outside of the adjustment tube segment 22 and fixedly connected to the second adjustment tube 23. When force is applied to the second pusher tube front seat 51, it moves the second adjustment tube 23 in the length direction of the adjustment tube segment 22.

The snap 52 is movably arranged on the second pusher tube front seat 51. One end faces the adjustment tube segment 22, and the other end faces away from the adjustment tube segment 22. The end facing the adjustment tube segment 22 has an inclined surface and is embedded in the snap slot 221 of the adjustment tube segment 22, matching the snap slot 221. The first adjustment screw 53 and the first spring 54 are installed on the second pusher tube front seat 51. The first adjustment screw 53 passes through the first spring 54 and connects to the snap 52. The first spring 54 applies an elastic force to the snap 52, allowing it to be embedded in the snap slot 221. When the second pusher tube front seat 51 drives the second adjustment tube 23 to move along the length direction of the adjustment tube segment 22, away from the puncture needle, the inclined surface of the snap 52 contacts the adjustment tube segment 22. This causes the snap 52 to move away from the adjustment tube segment 22, compressing the first spring 54, the buckle 52 is movable away from the currently engaged snap slot 221 at the adjustment tube segment 22. When the snap 52 moves toward the adjustment tube segment 22 and reaches the adjacent snap slot 211, the first spring 54 restores its deformation and drives the snap 52 to be embedded into the corresponding snap slot 221.

By lifting the first adjustment screw 53, the snap 52 can be made to disengage from the currently matched snap slot 221 toward the end facing the adjustment tube segment 22. This allows the second pusher tube front seat 51 to drive the second adjustment tube 23 to move along the length direction of the adjustment tube segment 22. The second adjustment tube 23 is equipped with a second pusher tube rear seat 55, which is positioned at the rear end of the second adjustment tube. The second pusher tube rear seat has a pushing surface at the rear end. The outer diameter or the outer circumscribed diameter of the pushing surface is greater than 10 mm, thereby increasing the contact area with the needle pulling pusher rod.

### Embodiment 4

The parts of this embodiment that are the same as in Embodiment 3 will not be repeated here. The differences are as follows:

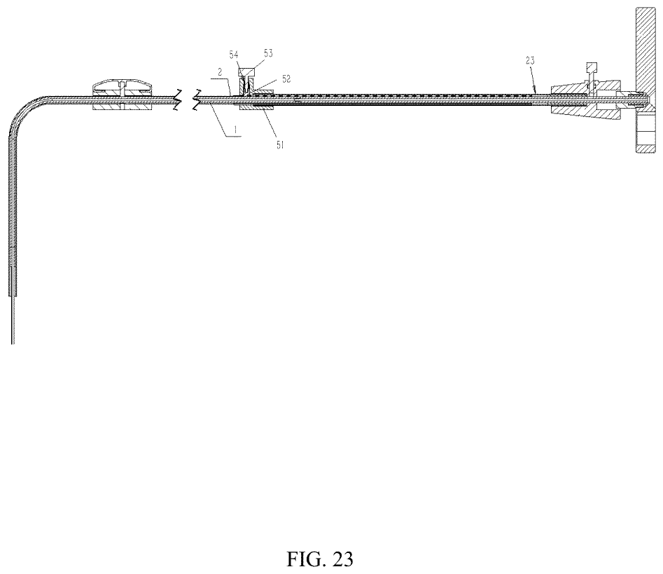

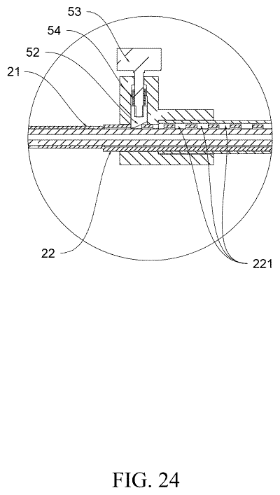

As shown in FIGS. 23-24, the main tube segment 21 and the adjustment tube segment 22 are an integrally molded structure. The middle adjustment segment is a second adjustment tube 23, which is sleeved on the outside of the adjustment tube segment 22. The second adjustment tube is controlled to move relative to the adjustment tube segment, allowing the total effective length of the second outer tube and the second adjustment tube to adaptively adjust based on the insertion depth of the needle. The second adjustment tube 23 is provided with a locking mechanism at the front end. The locking mechanism is used to fix the relative position between the second adjustment tube and the adjustment tube segment; the locking mechanism is the same as that in embodiment 3, and will not be repeated here.

### Embodiment 5

The parts of this embodiment that are the same as in Embodiment 2 will not be repeated here. The differences are as follows:

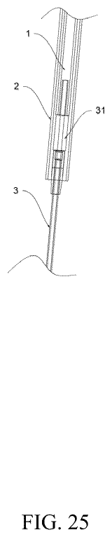

As shown in FIG. 25, the end of the second inner tube 1 is provided with a connector A 31 for quick connection with the puncture needle 3. The second inner tube is quickly connected to the puncture needle 3 via the connector A 31. The connection method of the connector A 31 includes one or more of threaded connection, latch connection, or adhesive bonding.

The connector A 31 can be threaded, glued, or locked to the puncture needle 3. When the connector A 31 connects to the second inner tube 1 or the puncture needle 3 via threading, the connector A 31 is a compact threaded connector, and the connector A 31 can rotate around its axis relative to the end of the second inner tube 1.

### Embodiment 6

The parts of this embodiment that are the same as in Embodiment 2 will not be repeated here. The differences are as follows:

The front end of the puncture needle 3 is provided with a resistance part, in the absence of an external force, the maximum internal circular diameter of the cross-segment of the resistance part is smaller than the outer diameter of the radioactive source, so that the radioactive source is unable to pass through the resistance part of the puncture needle by its own gravity alone, so as to avoid a fall of the radioactive source; in the event of the radioactive source being subjected to an external force, the resistance part is propped up by the radioactive source, so as to be able to provide for the passage of the radioactive source.

### Embodiment 7

The parts of this embodiment that are the same as in Embodiment 2 will not be repeated here. The differences are as follows:

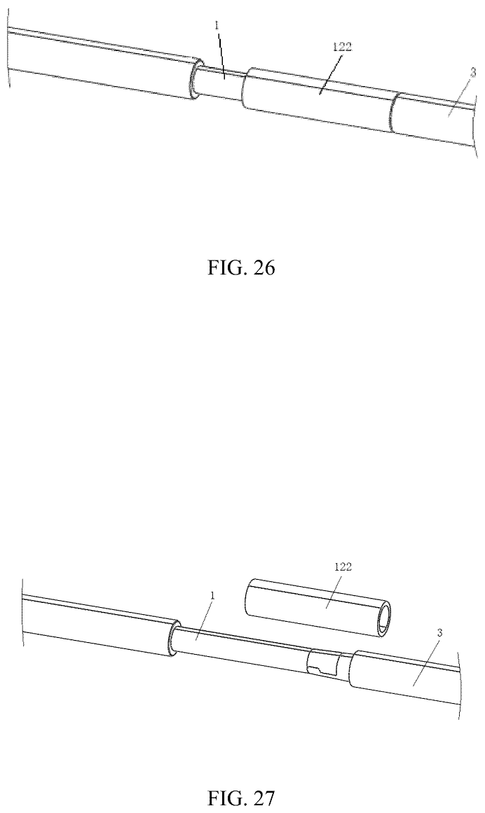

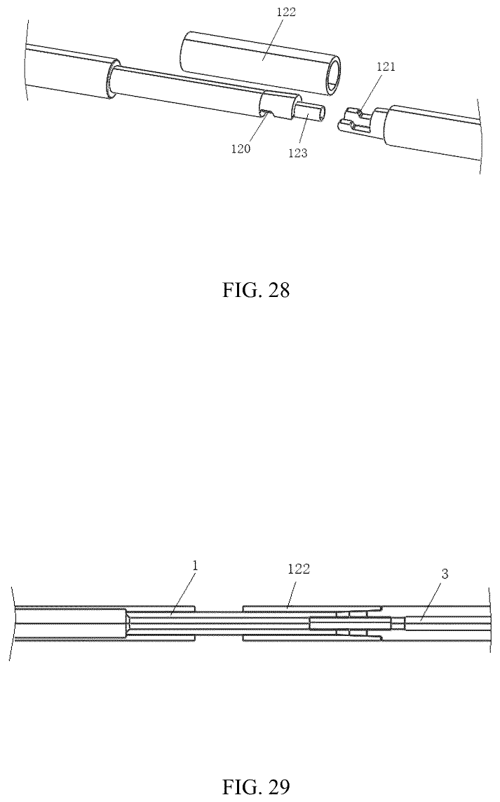

As shown in FIGS. 26-29, when the second inner tube 1 and the puncture needle 3 are connected by locking, the front end of the said second inner tube 1 is provided with a locking buckle A 120, the back end of the puncture needle is provided with a locking buckle B 121 which is interlocked and connected with the locking buckle A, and the second inner tube has a locking sleeve A 122 slidingly attached, and the outer diameter of the puncture needle 3 and the second inner tube 1 in the locking position is gradually increased in the direction from the second inner tube 1 to the puncture needle 3. The locking sleeve A 122 is slid into the locking position of the locking sleeve A and the locking sleeve B to be able to lock the second inner tube and the puncture needle tightly; at the same time, a connecting tube 123 is provided in the interior of the second inner tube and located at the position of the locking sleeve A, and the connecting tube 123 is located in the inner channel between the second inner tube 1 and the puncture needle 3 after the locking sleeve A and the locking sleeve B are locked, to ensure that the inner channel between the second inner tube 1 and the puncture needle 3 is continuous, avoiding the problem of the seed jamming at the joints of the interlocking clasp.

When the locking buckle A is fixedly provided at the front end of the inner tube, marking lines are provided on the front outer surface of the inner tube, on the rear outer surface of the inner tube, and on the rear outer surface of the puncture needle, respectively, to facilitate the user in determining the orientation of the locking buckle A 120 and the locking buckle B 121.

### Embodiment 8

The parts of this embodiment that are the same as in Embodiment 2 will not be repeated here. The differences are as follows:

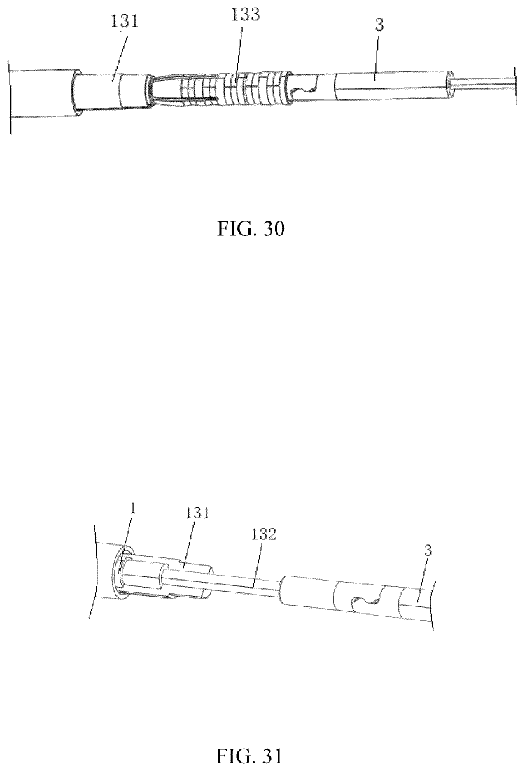

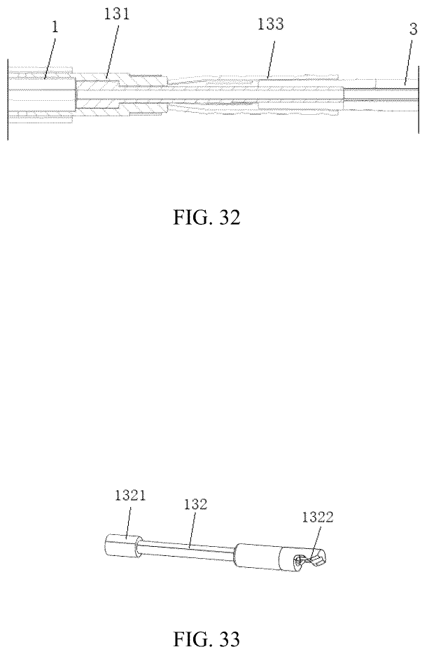

Specifically, as shown in FIGS. 30-33, the front end of the second inner tube 1 is connected to the puncture needle 3 using a free-spinning structure. This structure includes a fixed sleeve 131 and a free-spinning joint 132. The fixed sleeve 131 is fixed to the front end of the second inner tube 1, and inside it is a stepped surface, forming a free-spinning slot with the front face of the second inner tube 1. The free-spinning joint 132 has a connector B 1322 at its front end for quick connection to the puncture needle 3. The connection can use threads, locking clips, or adhesives. When the adapter 132 and the puncture needle 3 are connected via interlocking locking buckle, the front end of the adapter 132 is equipped with locking buckle C (which serves as connector B), while the rear end of the puncture needle 3 is provided with locking buckle D that interlocks with locking buckle C. A locking sleeve B 133 is slidably mounted on the adapter 132. By sliding the locking sleeve B 133 to the interlocked position of locking buckles C and D, the adapter 132 and puncture needle 3 can be securely locked together.

At this point, even if locking buckle C and locking buckle D are not aligned in orientation, the adapter 132 can be rotated to bring locking buckle C into the docking position with locking buckle D, facilitating their interlocking engagement.

### Embodiment 9

### a Sleeve-Type Radioactive Source Delivery Assembly's Usage Method:

Insert the puncture needle into the target body, fix the front end of the inner tube to the rear end of the puncture needle, or directly insert the puncture needle with the inner tube already connected into the target body, so that the front end of the outer tube contacts the target body. The other end of the inner tube is fixedly connected to the radioactive source implantation machine. The inner tube and outer tube can move relative to each other under the action of the needle pulling driver mechanism of the radioactive source implantation machine. The rear end of the inner tube is fixed, driving the rear end of the outer tube toward the target body side, so that the puncture needle moves away from the target body and is pulled out of the target body.

Move the main tube segment, which is sleeved on the outside of the inner tube, toward the target body, so that the front end of the main tube segment contacts the target body. Then, move the second adjustment tube backward, causing the second adjustment tube to move relative to the adjustment tube segment, and make the rear base of the second adjustment tube move closer to the radioactive source implantation machine. This increases the total effective length of the outer tube and locks the relative position between the second adjustment tube and the adjustment tube segment using the locking mechanism.

After fixing the other end of the inner tube to the radioactive source implantation machine, the needle pulling pusher rod pushes the rear base of the second adjustment tube to drive the outer tube to move toward the target body, thereby realizing the needle pulling action.

By using the flexible pushing wire, multiple radioactive sources are simultaneously pushed through the inner tube to the puncture needle. The flexible pushing wire moves forward, pushing the first radioactive source from the front end of the puncture needle into the target body, while the radioactive source implantation machine's needle pulling driver mechanism drives the inner tube and outer tube to move relative to each other, thus completing the needle pulling action. Then, after pulling the needle by a certain distance, the flexible pushing wire continues to move forward, pushing the second radioactive source from the front end of the puncture needle into the target body, while the radioactive source implantation machine's needle pulling driver mechanism drives the inner tube and outer tube to move relative to each other, completing the needle pulling action again. This process is repeated, with the needle pulling and implantation steps continuing until all radioactive sources are implanted into the target body at specific distances.

The above shows and describes the basic principles, main features, and advantages of the present invention. It is clear to those skilled in the art that the invention is not limited to the details of the exemplary embodiments mentioned above, and it can be implemented in other specific forms without departing from the spirit or essential characteristics of the invention. Therefore, regardless of the perspective, the embodiments should be regarded as illustrative and non-restrictive. The scope of the invention is defined by the appended claims rather than the description above. Hence, all modifications that fall within the meaning and range of equivalents of the claims should be included in the invention. No reference signs in the claims should be considered as limiting the scope of the claims.

Furthermore, although the description is provided according to specific embodiments, each embodiment does not necessarily contain only a single independent technical solution. This manner of description is merely for clarity. Those skilled in the art should consider the specification, and the technical solutions in the various embodiments may be appropriately combined to form other embodiments that can be understood by those skilled in the art.

We claim:

1. A sleeve-type radioactive source delivery assembly, characterized in that: it comprises an inner tube and an outer tube, wherein the outer tube is sleeved over the outside of the inner tube, the front end of the inner tube is connected to a puncture needle that is inserted into the target body, and the front end of the outer tube abuts against or connects to the target body; the inner tube and the outer tube are capable of relative movement under the action of a needle pulling driver, so that the puncture needle moves in a direction away from the target body and is withdrawn from the target body.

2. The sleeve-type radioactive source delivery assembly according to claim 1, characterized in that: the outer tube is made of a flexible, bendable, and deformable material, with the material being plastic; the inner diameter of the inner tube is 0.5 mm to 1.5 mm, and the outer diameter of the inner tube is 1.5 mm to 5 mm; the inner diameter of the outer tube is greater than the outer diameter of the inner tube.

3. The sleeve-type radioactive source delivery assembly according to claim 2, characterized in that: the portion of the inner tube near the puncture needle is made of a flexible, bendable, and deformable material, with the material being plastic, the portion of the inner tube away from the puncture needle is made of a rigid material, wherein the yield strength of the rigid material is greater than 50 MPa.

4. The sleeve-type radioactive source delivery assembly according to claim 1, characterized in that: the inner tube is directly connected to the puncture needle, with the puncture needle inserted into the front part of the inner tube and bonded and fixed thereto, a rigid guiding tube is sleeved over the front portion of the inner tube, the rear portion of the inner tube is a pushing guiding tube.

5. The sleeve-type radioactive source delivery assembly according to claim 1, characterized in that: the end of the inner tube is provided with a connector A for quick connection with the puncture needle, wherein the connection mode of the connector A is one or more of threaded connection, snap-fit connection, or adhesive bonding; when the inner tube and the puncture needle are connected via a snap-fit connection, the front end of the inner tube is provided with a snap-fit A, which serves as the connector A, and the rear end of the puncture needle is provided with a snap-fit B that interlocks and connects with snap-fit A, a locking sleeve A is slidably sleeved over the snap-fit A, and sliding the locking sleeve A to the interlocked position of snap-fit A and snap-fit B enables the inner tube and the puncture needle to be tightly locked together.

6. The sleeve-type radioactive source delivery assembly according to claim 5, characterized in that: when the snap-fit A is fixed at the front end of the inner tube, markings are respectively provided on the outer side surface of the front portion of the inner tube, the outer side surface of the rear portion of the inner tube, and the outer side surface of the rear portion of the puncture needle.

7. The sleeve-type radioactive source delivery assembly according to claim 1, characterized in that: the front end of the inner tube is connected to the puncture needle via a free-spinning structure, wherein the free-spinning structure includes a fixing sleeve and a free-spinning connector, the fixing sleeve is fixedly sleeved at the front end of the inner tube and is provided with a stepped surface, with a free-spinning slot formed between the stepped surface and the front end face of the inner tube, the rear portion of the free-spinning connector is provided with a shaft portion adapted to the shape of the free-spinning slot, which is rotatably disposed in the free-spinning slot, the front end of the free-spinning connector is provided with a connector B for quick connection with the puncture needle, and the connection mode of connector B is one or more of threaded connection, snap-fit connection, or adhesive bonding, when the free-spinning connector and the puncture needle are connected via snap-fit, the front end of the free-spinning connector is provided with a snap-fit C, which serves as connector B, and the rear end of the puncture needle is provided with a snap-fit D that interlocks and connects with snap-fit C, a locking sleeve B is configured to be slidably sleeved on the free-spinning connector, and sliding the locking sleeve B to the interlocked position of snap-fit C and snap-fit D enables the free-spinning connector and the puncture needle to be tightly locked together.

8. The sleeve-type radioactive source delivery assembly according to claim 1, characterized in that: the outer tube comprises an outer tube segment and a middle adjustment segment, wherein the middle adjustment segment is slidably sleeved and mounted on the outer tube segment.

9. The sleeve-type radioactive source delivery assembly according to claim 8, characterized in that: the outer tube segment comprises a main tube segment and an adjustment tube segment, wherein the adjustment tube segment is provided on one side of the main tube segment, and the middle adjustment segment is sleeved outside the adjustment tube segment, the middle adjustment segment is controlled to move relative to the adjustment tube segment and locked in place so that the total effective length of the outer tube can be adaptively adjusted according to the different insertion depths of the puncture needle, the main tube segment and the adjustment tube segment are integrally formed or connected together, and the middle adjustment segment is a second adjustment tube.

10. The sleeve-type radioactive source delivery assembly according to claim 9, characterized in that: a pushing tube base is provided on the second adjustment tube, with the pushing tube base located at the rear end of the second adjustment tube, the rear end of the pushing tube base is provided with a pushing surface, and the outer diameter or circumcircle diameter of the pushing surface is greater than 10 mm, thereby increasing the contact area with the needle pulling pusher.

11. The sleeve-type radioactive source delivery assembly according to claim 9, characterized in that: the front end of the second adjustment tube is provided with a locking mechanism, the locking mechanism being used to fix the relative position between the second adjustment tube and the adjustment tube segment.

12. The sleeve-type radioactive source delivery assembly according to claim 9, characterized in that: when the second adjustment tube and the outer tube segment move toward each other such that the total effective length of the outer tube is minimized, the connecting portion between the inner tube and the puncture needle is exposed from within the outer tube.

13. The sleeve-type radioactive source delivery assembly according to claim 11, characterized in that: a plurality of stop steps are provided on the outer side surface of the adjustment tube segment in its longitudinal direction, the stop steps are one or a combination of slots, snap holes, or protrusions; the locking mechanism comprises a stop head that cooperates with the stop steps to fix the relative position between the second adjustment tube and the adjustment tube segment, the stop head is one or a combination of an elastic snap block, a snap-fit, a latch, or a screw.

14. The sleeve-type radioactive source delivery assembly according to claim 13, characterized in that: when the stop steps are slots, the adjustment tube segment is an annular slot tube, and a plurality of annular slots are arranged in a linear array on its outer surface, with a spacing of 1 mm to 10 mm between adjacent annular slots, the locking mechanism is an elastic snap-fit, and the elastic snap-fit is provided with a latch that can be locked into the annular slot to fix the relative position between the second adjustment tube and the adjustment tube segment.

15. The sleeve-type radioactive source delivery assembly according to claim 1, characterized in that: the front end of the puncture needle is provided with a restrictive segment, the maximum inscribed circle diameter of the cross-section of the restrictive segment is smaller than the outer diameter of the radioactive source, so that the radioactive source cannot pass through the restrictive segment of the puncture needle under its own weight, thereby preventing it from dropping, when an external force is applied to the radioactive source, the restrictive segment is expanded by the radioactive source, allowing the radioactive source to pass through.

16. The sleeve-type radioactive source delivery assembly according to claim 15, characterized in that: the restrictive segment is one or a combination of a concave capillary tube, a variable diameter section, or resistance fillers, the resistance fillers are one or a combination of thin sheets, wire, or elastic body disposed inside the puncture needle, wherein the elastic body is one or a combination of polyurethane, silicone rubber, latex, or rubber.

17. A method for using a sleeve-type radioactive source delivery assembly, characterized in that: the puncture needle is inserted into the target body, and the front end of the inner tube is connected and fixed to the rear end of the puncture needle, or the puncture needle already connected to the inner tube is directly inserted into the target body, the front end of the outer tube is positioned against the target body, and the other end of the inner tube is fixedly connected to the radioactive source implantation system, under the action of the needle pulling driver of the radioactive source implantation system, the inner tube and outer tube are capable of relative movement, so that the puncture needle moves in the direction away from the target body and is withdrawn from it, the specific forms of said relative movement is that the rear side of the inner tube is fixed, and the rear side of the outer tube is driven to move toward the target body.

18. The method for using a sleeve-type radioactive source delivery assembly according to claim 17, characterized in that: the main tube segment sleeved on the outer side of the inner tube is pushed forward toward the target body, so that the front end of the main tube segment abuts against the target body, the second adjustment tube is then retracted, causing it to move relative to the adjustment tube segment and bringing the pushing tube base closer to the radioactive source implantation system, thereby increasing the total effective length of the outer tube, the locking mechanism is then used to fix the relative position between the second adjustment tube and the adjustment tube segment.

19. The method for using a sleeve-type radioactive source delivery assembly according to claim 17, characterized in that: after the rear end of the inner tube is fixedly connected to the radioactive source implantation system, a needle pulling pusher is used to push the pushing tube base, thereby driving the outer tube to move toward the target body to perform the needle withdrawal operation.

20. The method for using a sleeve-type radioactive source delivery assembly according to claim 17, characterized in that: multiple radioactive sources are simultaneously pushed through the inner tube into the puncture needle by means of a flexible pushing wire, as the flexible pushing wire moves forward and pushes the first radioactive source out from the front end of the puncture needle into the target body, the needle pulling driver of the radioactive source implantation system drives the inner and outer tubes to move relatively to perform the needle withdrawal action, the needle continues to be withdrawn for a specified distance, then, the flexible pushing wire continues to move forward, pushing the second radioactive source out from the front end of the puncture needle into the target body, while the needle pulling driver once again drives the inner and outer tubes to perform the needle withdrawal operation, the withdrawal and implantation process is repeated until all radioactive sources are implanted into the target body at predetermined distances.
